(19) United States
(12) Patent Application Publication
(10) Pub. No.: US 2008/0283422 A1
(43) Pub. Date: Nov. 20, 2008

(54) DENTAL CASE

(76) Inventor: John M. Jansheski, Maryville, TN (US)

Correspondence Address: LUEDEKA, NEELY & GRAHAM, P.C., P O BOX 1871, KNOXVILLE, TN, 37901, US

(21) Appl. No.: 11/748,795

(22) Filed: May 15, 2007

(51) Int. Cl. A61B 19/02

(52) U.S. Cl. 206/63.5

(57) ABSTRACT

A dental case useful for storing a dental guard. The case includes a base having a floor surrounded by an upstanding sidewall; a lid configured to fittingly overlie the base; and a tray suspended from the lid and configured to supportably receive the dental guard and suspend the dental guard above the floor of the base.

### FIELD

This disclosure relates to the field of storage cases for dental appliances. More particularly, this disclosure relates to a case for storing dental appliances such as mouth guards of the type used in the prevention of the grinding of teeth associated with Bruxism.

### BACKGROUND

Various types of dental mouth appliances are known. One example of a dental mouthguard is the type used for preventing Bruxism. Bruxism is generally characterized as the clenching and/or grinding of the teeth. Bruxism can cause chips and cracks in the teeth and wear of the biting surface. Bruxism typically occurs during sleep.

Dental appliances, such as Bruxism guards, are typically stored in plastic housings, with the guard sitting on the bottom or floor of the housing. It has been observed that this may result in unsanitary storage conditions, with the guard remaining in dampness. For example, even if the guard is rinsed before being placed in the housing, rinse water may remain on the guard and pool in the bottom of the housing, with the guard resting thereon.

What is needed is a case for storing dental appliances, such as Bruxism guards, which avoids disadvantages associated with conventional storage cases.

### SUMMARY

The above and other needs are met by a dental case useful for storing a dental guard of the type useful for inhibiting Bruxism.

In a preferred embodiment, the dental case includes a base having a floor surrounded by an upstanding sidewall; a lid configured to fittingly overlie the base; and a tray suspended from the lid and configured to supportably receive the dental guard and suspend the dental guard above the floor of the base.

The case advantageously suspends the guard above the floor of the base to facilitate drainage and/or air circulation, thereby avoiding unsanitary storage conditions associated with conventional cases. Also, when the lid is opened the tray is inclined substantially parallel to the lid so as to enable easy access to the guard for insertion or removal thereof relative to the case.

Additionally, if desired, the lid may be configured to be locked open relative to the base, as by engaging the catch and the receiver, the tray remains inclined to facilitate drainage and/or air circulation.

If desired, the tray may also be configured to be removably suspended from the lid so that it may be readily removed from the case to facilitate washing thereof and of the remaining components of the case.

### DESCRIPTION OF THE DRAWINGS

Further advantages of the disclosure are apparent by reference to the detailed description in conjunction with the figures, wherein elements are not to scale so as to more clearly show the details, wherein like reference numbers indicate like elements throughout the several views, and wherein:

FIG. 1 is a frontal perspective view of a dental case in an open orientation and in accordance with a preferred embodiment of the disclosure.

FIG. 2 is a rear perspective view of the dental case in a closed orientation.

FIG. 3 shows installation/removal of a dental appliance from the dental case of FIG. 1

FIGS. 4 and 5 show a base component of the dental case of FIG. 1.

FIGS. 6 and 7 show a lid component of the dental case of FIG. 1.

FIGS. 8 and 9 show a tray component of the dental case of FIG. 1.

### DETAILED DESCRIPTION

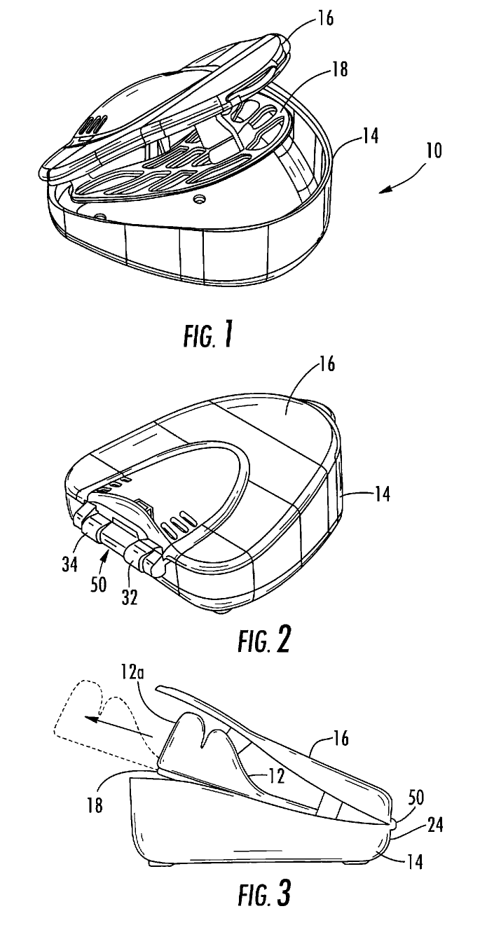

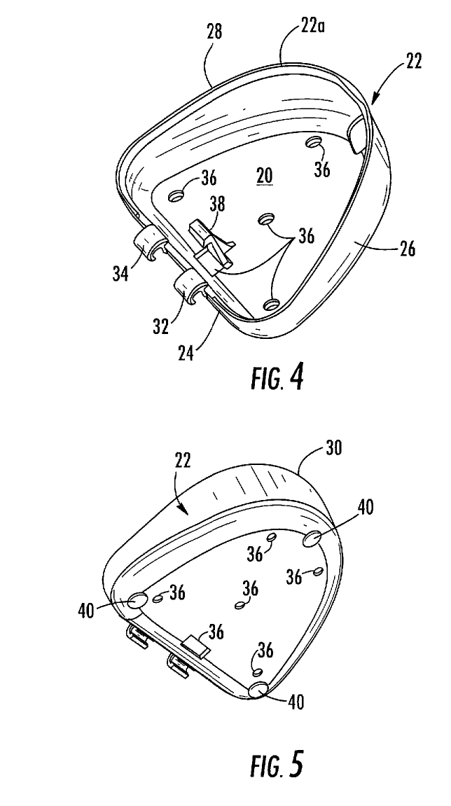

With initial reference to FIGS. 1-3, the disclosure relates to a dental case 10 useful for storing a dental guard 12 (FIG. 3) of the type useful for inhibiting Bruxism. In a preferred embodiment, the case 10 includes a base 14, a lid 16, and a tray 18.

The components of the dental case 10 are each desirably of one-piece molded plastic construction and configured to be erected into the case 10 by snap-fit or friction-fit construction.

The dental guard 12 is characterized as having a generally U-shaped configuration having a frontal apex portion and a pair of opposite free ends. In this regard, examples of preferred Bruxism guards with which the dental case 10 may be used are described in U.S. patent application Ser. No. 11/608,274, filed Dec. 8, 2006, and entitled DENTAL GUARD, and U.S. patent application Ser. No. 11/672,683, filed Feb. 8, 2006, and entitled DENTAL GUARD, incorporated herein by reference in their entireties.

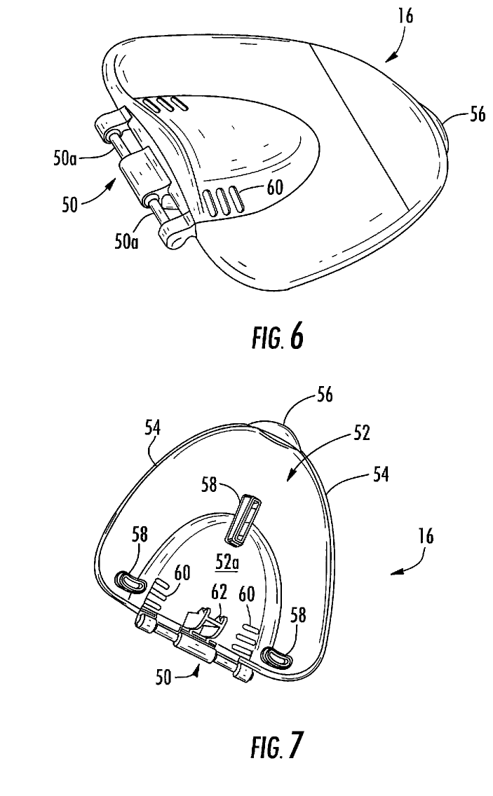

With additional reference to FIGS. 4-5, the base 14 includes a generally rounded triangular-shaped floor 20 surrounded by an upstanding sidewall 22. The uppermost edge 22a of the sidewall 22 has a reduced thickness to mate with a corresponding lip structure of the lid 16 described more fully below. A rear portion 24 of the sidewall 22 has a substantially uniform height, with forward portions 26 and 28 tapering upwardly from the rear portion 24 to an apex 30 at the front of the base 14.

A pair of spaced apart female hinge members 32 and 34 are defined along the rear portion 24. A plurality of vents 36 or other openings are defined through the floor 20 for facilitating drainage of fluids and/or air circulation. A catch 38 extends upwardly from the floor 20 adjacent the rear portion 24 of the sidewall 22 for releasably engaging a portion of the lid 16 to support the lid 16 in an open relationship to the base 14 as described more fully below. Feet 40 may be defined on the bottom of the base 14 to raise the base 14 from a supporting surface, such as a table, to further enhance drainage and/or air circulation.

With additional reference to FIGS. 6-7, the lid 16 is generally of a rounded triangular shape so as to fittingly overlie the base 14. The lid 16 includes male hinge member 50 configured to cooperate with the female hinge members 32 and 34 so as to hingedly connect the lid 16 and the base 14 at a rear portion of the case 10. Frictional interaction of the hinge members preferably provides sufficient pressure to maintain the lid 16 in a selected relationship to the base 14, i.e., open or closed, regardless of the orientation of the case 10, e.g., upside down, upright. As will be appreciated, this also advantageously avoids the need for latching structure to maintain the lid in a closed orientation.

As shown, the hinge member 50 has rods 50a that snap-fit into the members 32 and 34. The lid 16 has an interior surface 52 that includes a lip 54 that extends around the circumference of the lid 16 and engages an upper interior portion of the sidewall 22 of the base 14 when the lid 16 is closed upon the base 14 so as to mate with the edge 22a of the sidewall 22. In this regard, installation of the lid 16 onto the base 14 provides an enclosed housing.

A central portion 52a of the surface 52 is preferably domed so as to provide a raised area underneath the lid 16. The lid 16 may include a tab 56 proximate the apex or front region of the lid 16 configured for a user to engage to engage the front of the lid 16. The interior surface 52 also includes a plurality of mounts 58 for releasably mounting of the tray 18 adjacent the interior surface 52. One or more vents 60 are preferably defined through the central portion 52a for enhancing air circulation. A receiver 62 is defined adjacent the hinge member 50 for cooperating with the catch 38 of the base 14 to releasably lock the lid 16 in an open relationship with the base 14 as may be desired.

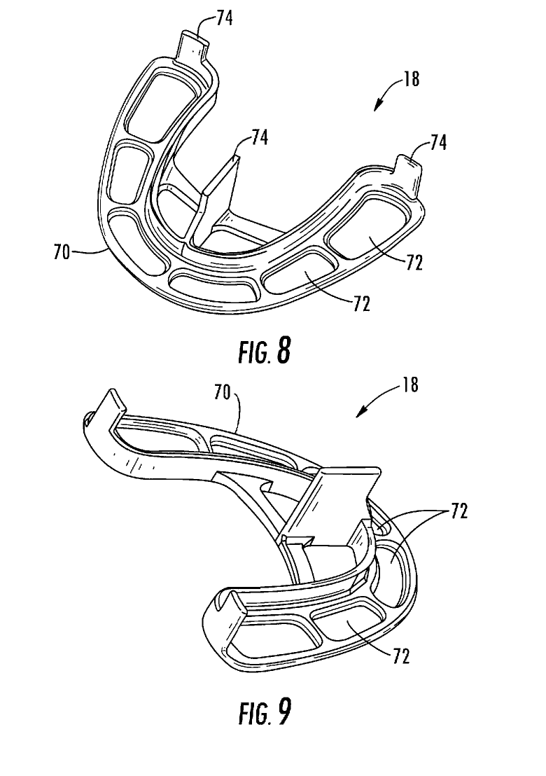

With additional reference to FIGS. 8-9, the tray 18 is configured to receive a dental appliance, such as the dental guard 12. In this regard, the tray 18 is sized to fit within the case 10 underneath the lid 14 so as to reside in the enclosed housing provided by the lid 16 and the base 14. The tray 18 includes a support surface 70 configured generally in the shape of the guard 12 or other dental appliance. For example, the support surface 70 is shown configured to receive the U-shaped guard 12 and is therefore generally U-shaped in configuration. As seen, the front or apex 70a of the surface 70 is adjacent the front or apex 12a of the guard 12.

To promote drainage and/or air circulation, the support surface 70 includes a plurality of vents 72. A plurality of hangers 74 extend upwardly from the tray 18 and are configured for being releasably received by the mounts 58. For example, the mounts 58 are shown as blind elongate slots and the hangers 74 as projections that fittingly engage the slots. The hangers 74 are preferably sized and located to suspend the tray 18 from the lid 16 so that the support surface 70 is substantially parallel to the general plane of the lid 18. However, as will be appreciated, the mounts 58 could alternatively be provided on the tray 18 and the hangers 74 on the lid 16.

The tray 18 is oriented so that the apex 70a of the surface 70 faces the apex or front 30 of the base 14 and the front or apex of the lid 16. Thus, the guard 12 may be installed so that the corresponding apex or front of the guard 12 is oriented adjacent the front or apex of the lid 16 and the base 14.

The cooperation of the hangers 74 and the mounts 58 enable the tray 18 to be removably suspended from the lid 16. This offers several advantages. For example, in one aspect the tray 18 suspends the guard 12 above the floor 20 to facilitate drainage and/or air circulation, thereby avoiding unsanitary storage conditions associated with conventional cases. However, it will be understood that the tray 18 may also be non-removably suspended from the lid 16. For example, the tray 18 may be sonic-welded, adhesively secured, or otherwise fixedly adhered to the lid 16.

Also, as seen in FIGS. 1 and 3, when the lid 16 is opened the tray 18 is inclined substantially parallel to the lid 16 so as to enable easy access to the guard 12 for insertion or removal thereof relative to the case 10. As will be appreciated, the orientation of the apex or front of the guard 12 with the front or apex of the base 14 and the lid 16 facilitates removal and installation.

Additionally, the tray 18 may be inclined as by opening the lid 16 to facilitate drainage and/or air circulation. For example, the inclination positions the apex or front of the guard 12 (which is a barrier to drainage) at an elevated position so that the free or exposed ends of the guard 12 are at lower positions. In addition, the tray 18 may be readily removed from the case 10, as by pulling on the tray 18 to disengage the hangers 74 from the mounts 58, to facilitate washing thereof and of the remaining components of the case.

The foregoing description of preferred embodiments for this disclosure have been presented for purposes of illustration and description. They are not intended to be exhaustive or to limit the disclosure to the precise form disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

What is claimed is:

1. A dental case useful for storing a dental guard of the type used for inhibiting Bruxism, the case comprising:
a base comprising a U-shaped floor surrounded by an upstanding sidewall and having a frontal apex;
a lid configured to fittingly overlie the base and having a frontal apex; and
a tray suspended from the lid such that a frontal apex of the tray is aligned with the frontal apexes of the base and the lid, the tray being configured to supportably receive the dental guard and suspend the dental guard above the floor of the base.

2. The dental case of claim 1, wherein the base, the lid, and the tray each include a plurality of vent apertures.

3. The dental case of claim 1, wherein the lid and the base are hingedly connected to one another adjacent a rear portion of the case so that the frontal apexes of the base and the lid are urged away from one another to open the case, and wherein when the case is in the opened orientation the tray is inclined substantially parallel to the lid.

4. The dental case of claim 1, lid and the base include a cooperating catch and release to enable the lid to be releasably locked in an inclined orientation relative to the base.

5. The dental case of claim 1, wherein the lid and the tray include cooperating hangers and mounts to enable the tray to be removably suspended from the lid.
